(19) United States
(12) Patent Application Publication
(10) Pub. No.: US 2007/0208409 A1
(43) Pub. Date: Sep. 6, 2007

(54) Flexible stent-graft devices and methods of producing the same

(75) Inventor: Fergus Quigley, Waltham, MA (US)

Correspondence Address: HOFFMANN & BARON, LLP, 6900 JERICHO TURNPIKE, SYOSSET, NY, 11791, US

(73) Assignee: BOSTON SCIENTIFIC SCIMED, INC.

(21) Appl. No.: 11/365,324

(22) Filed: Mar. 1, 2006

(51) Int. Cl. A61F 2/88

(52) U.S. Cl. 623/1.13; 623/1.22; 264/259

(57) ABSTRACT

An implantable stent-graft (10) includes a radially distensible, tubular stent (20) having opposed open ends (12′, 14′) and a stent wall structure (16′) having opposed exterior and luminal surfaces (30, 32); and a segmented, non-textile polymeric tubular structure (16) including a plurality of graft segments (18) circumferentially disposed about one of the stent surfaces (30, 32), the graft segments (18) including first portions (24) securably disposed to the one the stent surfaces (30, 32) and second portions (26) not securably disposed to the one the stent surfaces (30, 32); wherein adjacent graft segments (18) are disposed over one and the other to define overlaps (26′), the overlaps (26′) forming a fluid tight seal when implanted in a body lumen.

### FIELD OF THE INVENTION

The present invention relates to flexible stent-graft devices and methods for making the same. More particularly, the present invention relates to a radially distensible stent and a segmented, non-textile polymeric tubular structure having a plurality of graft segments circumferentially disposed over the stent.

### BACKGROUND OF THE INVENTION

An intraluminal prosthesis is a medical device used in the treatment of diseased bodily lumens. One type of intraluminal prosthesis used in the repair and/or treatment of diseases in various body vessels is a stent. A stent is generally a longitudinal tubular device formed of biocompatible material which is useful to open and support various lumens in the body. For example, stents may be used in the vascular system, urogenital tract, esophageal tract, tracheal/bronchial tubes and bile duct, as well as in a variety of other applications in the body. These devices are implanted within the vessel to open and/or reinforce collapsing or partially occluded sections of the lumen.

Stents generally include an open flexible configuration. This configuration allows the stent to be inserted through curved vessels. Furthermore, this configuration allows the stent to be configured in a radially compressed state for intraluminal catheter implantation. Once properly positioned adjacent the damaged vessel, the stent is radially expanded so as to support and reinforce the vessel. Radial expansion of the stent may be accomplished by inflation of a balloon attached to the catheter or the stent may be of the self-expanding variety which will radially expand once deployed. Tubular shaped structures, which have been used as intraluminal vascular stents, have included helically wound coils which may have undulations or zig-zags therein, slotted stents, ring stents, braided stents and open mesh wire stents, to name a few. Super-elastic materials and metallic shape memory materials have also been used to form stents.

A graft is another commonly known type of intraluminal prosthesis which is used to repair and replace various body vessels. A graft provides a lumen through which fluids, such as blood, may flow. Moreover, a graft is often configured as being generally impermeable to blood to inhibit substantial leakage of blood therethrough. Grafts are typically hollow tubular devices that may be formed of a variety of materials, including textile and non-textile materials.

A stent and a graft may be combined into a stent-graft endoprosthesis to combine the features and advantages of each. For example, tubular coverings have been provided on the inner and/or outer surfaces of stents to form stent-grafts. It is often desirable to use a thin-walled graft or covering in the stent-graft endoprosthesis to minimize the profile of the endoprosthesis and to maximize the flow of blood through the endoprosthesis. In such cases non-textile materials, such as polymeric tubes or sheets formed into tubes, are often used. Expanded polytetrafluoroethylene or e-PTFE is one common polymeric material used as the graft portion or covering of a stent-graft endoprosthesis. Expanded polytetrafluoroethylene grafts, however, are subject to plastic deformation, especially when, for example, compressing the stent-graft for loading into its delivery system, delivering the stent-graft through a highly tortuous bodily lumen and/or placing or deploying the stent-graft at the target implant site. Such plastic deformation may lead to the tearing of the ePTFE, leaving the stent-graft endoprosthesis prone to leakage of blood therethrough. Furthermore, plastic deformation of expanded polytetrafluoroethylene grafts may lead to physical deformities in the graft, such as buckling, which is also undesirable because it may lead to poor blood flow patterns.

Sheets or films of ePTFE have been used to cover or line stents. For example, U.S. Pat. Nos. 5,700,285 and 5,735,892 to Myers et al. describe overlapping a sheet of ePTFE onto a stent to form a tubular graft. The graft is secured to the stent by an application of thermoplastic adhesive and heat treatment to melt the adhesive. A seam, which is formed where the sheet overlaps, is also sealed through the use of the thermoplastic adhesive. Such stent-grafts having a unitary tubular ePTFE covering adhesively secured to the stent, however, do not have flexibility associated with the graft to avoid plastic deformation of the graft when subjected to certain stresses, such as bending stresses during delivery through tortuous bodily lumens.

U.S. Pat. No. 6,264,684 to Banas et al. describes a helically supported ePTFE graft, i.e., a stent-graft. The support or stent wire is encapsulated in an ePTFE strip. The strip is helically wound over a mandrel into a configuration having adjacent windings forming overlapping regions. The overlapping regions are secured to one and the other through the use of a thermoplastic adhesive and a heat treatment for melting the thermoplastic adhesive. U.S. Pat. No. 6,790,225 to Shannon et al. describes the helically winding of ePTFE tape to completely cover a stent and sintering the tape to the stent. Any overlaps of the ePTFE tape are also sintered together. Such unitary ePTFE graft structures also lack sufficient flexibility necessary to avoid plastic deformation of the graft when subjected certain stresses, such as bending stresses during delivery through tortuous bodily lumens, lacks flexibility.

U.S. Pat. No. 6,520,986 to Martin et al. described the securement or interweaving of ePTFE graft strips through helical windings of an undulating stent wire. The ePTFE strips are spaced apart from the apices of the undulating wire such that no strip completely covers a winding of the undulating wire. The graft strips are secured to the stent wire by use of a thermoplastic adhesive and the application of heat. While such a resulting stent-graft may have additional flexibility as compared to the above-described stent-grafts, the graft wall is non-continuous, thereby not providing by it self a fluid tight graft wall.

U.S. patents and U.S. Patent Application Publications Nos. 6,287,335 to Drasler et al.; U.S. Pat. No. 6,551,350 to Thornton et al.; 2004/0019375 to Casey et al. and 2004/0033364 to Spiridigliozzi et al. describe the use of folds, folded flaps, pleats and/or crimps to improve the flexibility of grafts, including ePTFE stent-grafts. Such folds folded flaps, pleats and/or crimps, however, increase the overall profile of the device.

U.S. Pat. No. 6,344,054 to Parodi describes a stent graft having its graft being secured to only one end of the stent. Such a graft avoids undue stresses being placed on the graft during contraction and expansion of the stent by only securing one end of the graft to the stent.

U.S. Patent Application Publication No. 2003/0220682 to Kujawski describes a hybrid braided stent having a plurality of overlapping graft segments. The graft segments are described as being textile graft segments made by, for example, braiding yarns. One end of a graft segment is secured to the stent, and the other end of the graft segment overlaps an adjacent secured graft segment.

Thus, there is a need for a stent-graft having a polymeric, non-textile graft that has enhanced flexibility.

### SUMMARY OF THE INVENTION

In one aspect of the present invention, a endoprosthesis or stent-graft is provided. The stent graft includes a radially distensible, tubular stent including opposed open ends and a stent wall structure having opposed exterior and luminal surfaces; and a segmented, non-textile polymeric tubular structure including a plurality of graft segments circumferentially disposed about at least one of the stent surfaces, the graft segments including first portions securably disposed to the one the stent surface and second portions not securably disposed to the one the stent surface. The adjacent graft segments may be disposed over one and the other to define overlaps, and these overlaps form a fluid tight seal when implanted in a body lumen. For the purposes of this invention, the terms tubular graft and tubular covering may be used interchangeably.

The second portion of one of the graft segments may be disposed over the first portion of the adjacent graft segment. The second portion of the graft segment may slidingly abut the first portion of the adjacent graft segment. Desirably, the graft segments may be disposed over the exterior surfaces of the stent.

The stent-graft of this aspect of the present invention may further include a second non-textile polymeric tubular graft structure securably disposed about the luminal surfaces of the stent. Desirably, the first portions of the graft segments may be securably attached to portions of the second graft structure. Desirably, the second portions of the graft segments may not be securably attached to portions of the second graft structure. Desirably, the graft segments may be substantially unfolded segments.

Desirably, the stent further includes a longitudinal length, wherein the longitudinal length remains substantially constant upon radial expansion or radial contraction of the stent. The stent may be a self-expanding stent, a balloon-expandable stent or combinations thereof. The stent may also include a plurality of undulating stent segments.

Desirably, the stent includes an undulating wire helically wound into a plurality of circumferential windings to define the stent wall structure. The first portion of the graft segment may be securably attached to at least one of the circumferential windings of the undulating wire. Further, the first portion of the graft segment may be securably attached to at least two of the circumferential windings of the undulating wire. Moreover, the overlap of the adjacent graft segments longitudinally extends over at least one of the circumferential windings of the undulating wire.

Desirably, the stent includes a biocompatible material selected from the group consisting of metallic materials, polymeric materials, bioabsorbable materials, biodegradable materials, and combinations thereof.

The graft segments may include polymeric graft material selected from the group consisting of polyesters, polypropylenes, polyethylenes, polyurethanes, polynaphthalenes, polytetrafluoroethylenes, expanded polytetrafluoroethylenes, silicones, and combinations and copolymers thereof. Desirably, the graft segments include expanded polytetrafluoroethylene. Desirably, the graft segments are extruded, cast, spun or molded polymeric segments.

The second tubular graft may include polymeric graft material selected from the group consisting of polyesters, polypropylenes, polyethylenes, polyurethanes, polynaphthalenes, polytetrafluoroethylenes, expanded polytetrafluoroethylenes, silicones, and combinations and copolymers thereof. Desirably, the second tubular graft includes expanded polytetrafluoroethylene.

In another aspect of the present invention, a stent-graft includes a radially distensible, tubular stent having opposed open ends including an undulating wire helically wound into a plurality of circumferential windings to define stent wall structure having opposed exterior and luminal surfaces; a non-textile, polymeric tubular graft structure securably disposed about the luminal surface of the stent; and a segmented, non-textile polymeric tubular structure including a plurality of graft segments circumferentially disposed about the exterior surface of the stent, the graft segments including first portions securably disposed to the exterior surface of the stent and second portions not securably disposed the exterior surface of the stent; wherein adjacent graft segments may be disposed over one and the other to define overlaps, the overlaps forming a fluid tight seal when implanted in a body lumen.

In another aspect of the present invention, a method of making a stent-graft is provided. The method includes the steps of providing a radially distensible, tubular stent having opposed open ends including an undulating wire helically wound into a plurality of circumferential windings to define stent wall structure having opposed exterior and luminal surfaces; providing a first non-textile, polymeric graft strip; helically winding the first strip over at least one of the circumferential windings of the undulating wire of the exterior stent surface to define a first juxtaposed strip-stent region; providing a second non-textile, polymeric graft strip; helically winding the second strip over at least one of the circumferential windings of the undulating wire of the exterior stent surface to define a second juxtaposed strip-stent region and over a portion of the first strip to define an overlap of the first and the second strips; securing portions of the first and second strips at the first and the second juxtaposed regions to the stent; and not securing the graft strips to one and the other at the overlap.

Desirably, the step of securing portions of the first and second strips at the first and the second juxtaposed regions to the stent further includes laminating the strips to the stent regions. Desirably, the step of not securing the graft strips to one and the other at the overlap includes masking the strips at the overlap so that the strips at the overlap are not laminated to one and the other.

The method of the making the stent-graft may further include the steps of providing a non-textile, polymeric tubular graft disposed about the luminal surface of the stent; and securing the graft to the luminal surfaces of the stent. In this aspect, the method may further include the step of securing the graft to the first and the second strips.

In another aspect of the present invention, a radially distensible, tubular stent having opposed open ends to define a length therebetween is provided. The stent includes an undulating wire helically wound into a plurality of circumferential windings to define a stent wall structure having opposed exterior and luminal surfaces. Desirably, the undulating wire includes a series of peaks and valleys along its length. Desirably, the peaks may be angularly offset from the longitudinal length of the stent. The peaks may be angularly offset from about 1° to about 45° from the longitudinal length of the stent.

### BRIEF DESCRIPTION OF THE DRAWINGS

FIG. 1 is a perspective view of a segmented stent-graft according to the present invention.

FIG. 2 is an expanded, partial cross-sectional view of the stent-graft of FIG. 1 taken along the 2-2 axis.

FIG. 3 is a partial exploded view of the stent-graft of FIG. 2 further detailing the overlapping of the graft segments of the present invention.

FIG. 4 is a partial exploded view of the stent-graft of FIG. 2 further detailing an alternate aspect of the overlapping of the graft segments of the present invention.

FIG. 5 is a side elevational view of the stent-graft of FIG. 2 being in a bent position.

FIG. 6 is a partial cross-sectional view of the stent-graft of FIG. 5 showing sliding disengagement of overlapping graft segment portions.

FIG. 7 is a partial exploded view of the stent of FIG. 2 further detailing the stent configuration.

FIG. 8 is a partial exploded view of the stent of FIG. 2 further detailing an alternate stent configuration.

FIG. 9 is a partial exploded view of the stent of FIG. 2 further detailing yet another alternate stent configuration.

FIG. 10 is a partial exploded view of the stent of FIG. 2 further detailing yet another alternate stent configuration.

FIG. 11 is a cross-sectional view of the stent-graft of FIG. 2 taken along the 11-11 axis.

FIG. 12 is a cross-sectional view of the stent-graft of FIG. 2 taken along the 12-12 axis.

FIG. 13 is a longitudinal view of a wire stent of the present invention.

FIG. 14 is a longitudinal view of a zig-zag stent of the present invention.

FIG. 15 is a perspective view of slotted stent of the present invention.

FIG. 16 is a perspective view of a helical coil stent formed of a single wound wire according to the present invention.

FIG. 17 is a perspective view of a stent having an elongate pre-helically coiled configuration according to the present invention.

### DETAILED DESCRIPTION OF THE PREFERRED EMBODIMENT

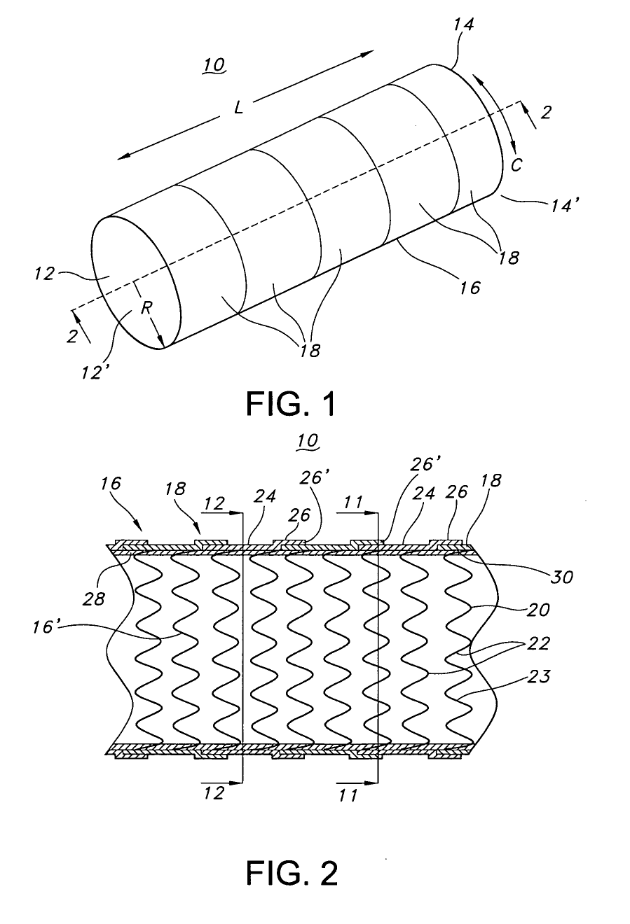

FIG. 1 is a perspective view of the segmented stent-graft 10 of the present invention. The segmented stent-graft 10 is a hollow, tubular structure or device having opposed open ends 12, 14. The stent-graft 10 includes a tubular wall 16 disposed between the open ends 12, 14. As depicted in FIG. 1, the tubular wall 16 extends along the longitudinal direction, which is depicted as the L-axis, of the stent-graft 10. The tubular wall 16 includes a plurality of overlapping graft segments 18. The graft segments 18 extend around the circumference, which is indicated by the C-axis or C-vector, of the stent-graft 10. The R-axis defines a radial extent of the stent-graft 10 of the present invention. As depicted in FIG. 1, stent-graft 10 is a substantially longitudinally straight tubular device, but the present invention is not so limited. Stent-graft 10 may have a varying radial extent, for example, a varied diameter, outwardly or inwardly flared extents, and the like

FIG. 2 is an expanded, partial cross sectional view of the stent-graft 10 of FIG. 1 taken along the 2-2 axis. As depicted in FIG. 2, the stent-graft 10 may include a stent 20. Various stent types and stent constructions may be employed in the invention as the stent 20. Among the various stents useful include, without limitation, self-expanding stents and balloon expandable extents. The stents may be capable of radially contracting, as well and in this sense can best be described as radially distensible or deformable. Self-expanding stents include those that have a spring-like action which causes the stent to radially expand, or stents which expand due to the memory properties of the stent material for a particular configuration at a certain temperature. Nitinol is one material which has the ability to perform well while both in spring-like mode, as well as in a memory mode based on temperature. Other materials are of course contemplated, such as stainless steel, platinum, gold, titanium and other biocompatible metals, as well as polymeric stents. The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like. Examples of various stent configurations are shown in U.S. Pat. No. 4,503,569 to Dotter; U.S. Pat. No. 4,733,665 to Palmaz; U.S. Pat. No. 4,856,561 to Hillstead; U.S. Pat. No. 4,580,568 to Gianturco; U.S. Pat. No. 4,732,152 to Wallsten, U.S. Pat. No. 4,886,062 to Wiktor, and U.S. Pat. No. 5,876,448 to Thompson, all of whose contents are incorporated herein by reference.

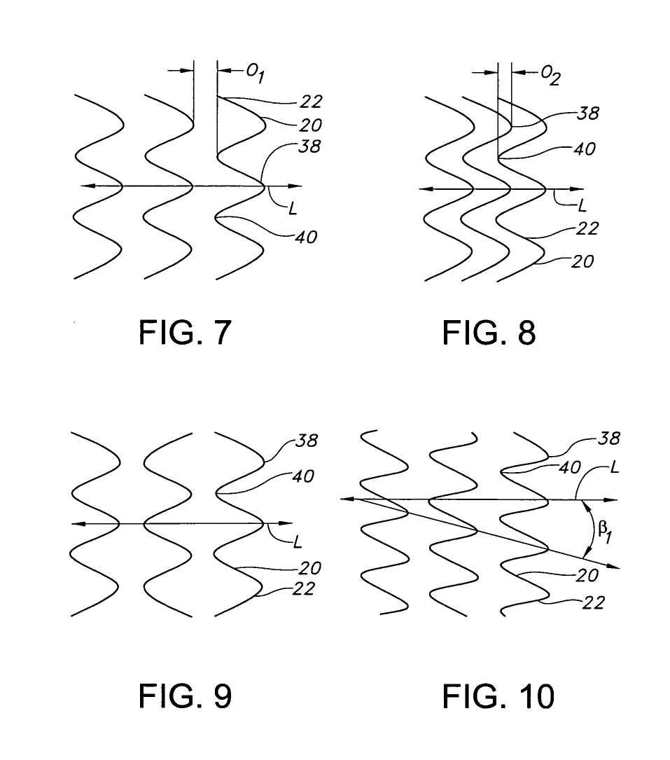

Desirably, stent 20 is one that has minimal foreshortening, i.e., a stent wherein its longitudinal length remains substantially constant upon radial expansion or radial contraction of the stent. As depicted in FIG. 2, such a stent 20 having minimal foreshortening may include undulating stent portions 22. Such undulating stent portions 22 will be further described in conjunction with the description of FIGS. 7-10.

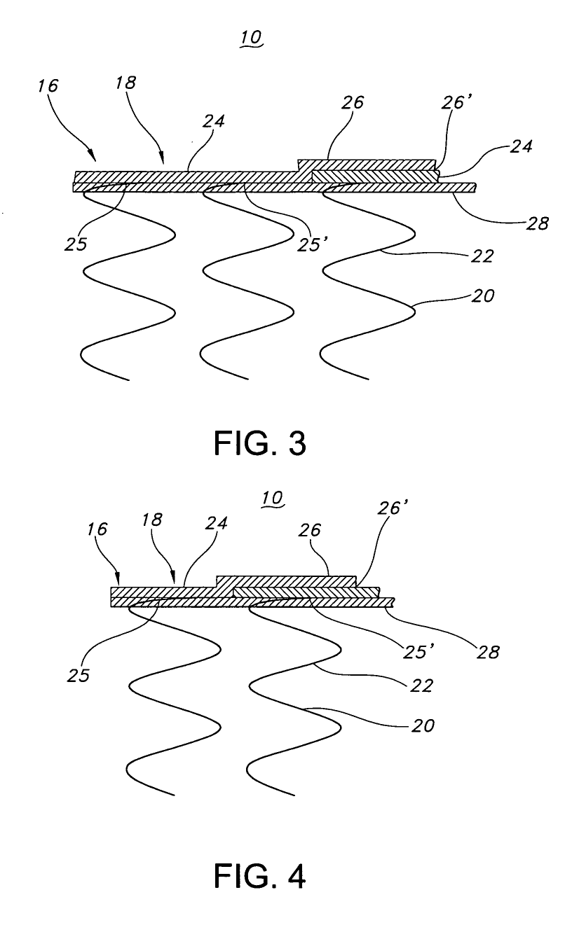

As depicted in FIGS. 2-3, the tubular wall 16 includes graft segments 18 that may slidingly overlap one and the other. For example, the graft segments 18 may include a first portion 24 securably disposed to one or more undulating stent portions 22 of the stent 20 and a second portion 26 which is not secured to the undulating stent portions 22. The second portion 26 of the graft segment 18 is slidably disposed or juxtaposed over the first portion 24 of an adjacent graft segment 18. Although the graft segments 18 are depicted as being disposed over the exterior surfaces 30 of the stent 20, the present invention is not so limited. For example, the graft segments 18 may be disposed over interior or luminal surfaces 32 of the stent 22. Desirably, the graft segments 18 are unfolded graft segments, i.e., the segments are not bent or doubled up so that one part lies on another part of the same segment.

While the first portion 24 of the graft segment 18 is depicted in FIGS. 2-3 as being secured to two undulating stent portions 22 of the stent 20, the present invention is not so limited. The first portion 24 of the graft segment 18 may suitably be secured to at least one or more of the adjacent undulating stent portions 22 of the stent 20. Desirably, as depicted in FIG. 4, the first portion 24 of the graft portion 18 is secured to at least one of the undulating stent portions 22. Further, while the second portion 26 of the graft segment 18 is depicted as slidingly overlapping one of the undulating stent portions 22, the present invention is not so limited. Desirably, the second portion 26 of the graft segment 18 overlaps at least one or more of the undulating stent potions 22. The present invention is not limited to any particular number undulating stent portions 22 having the first portions 24 secured thereto or having the second portions 26 slidingly juxtaposed thereover. In general, the flexibility of the stent-graft 10 of the present invention may increase with an increasing number of the graft segments 18. The flexibility of the stent-graft 10 of the present invention may also increase with decreasing number of undulation stent portions 22 to which the first portions 24 are secured thereto.

The stent-graft 10 of the present invention may optionally include a second tubular graft structure 28. The second tubular graft structure 28 may be a continuous tubular structure or a segmented tubular structure similar to one having graft segments 18.

Desirably, the stent 20 is made from any suitable implantable, biocompatible, bioabsorbable or biodegradable material, including without limitation nitinol, stainless steel, cobalt-based alloy such as Elgiloy®, platinum, gold, titanium, tantalum, niobium, polymeric materials and combinations thereof. Useful and nonlimiting examples of polymeric stent materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), poly(phosphate ester) and the like.

Further, the stent 22 may have a composite construction, such as described found in U.S. Patent Application Publication 2002/0035396 A1, the contents of which is incorporated herein by reference. For example, the stent 22 may have an inner core of tantalum gold, platinum, iridium or combination of thereof and an outer member or layer of nitinol to provide a composite wire for improved radiocapacity or visibility. Alternatively, a radiopaque member or wire may be secured to a portion of the stent 20 for improved radiocapacity or visibility.

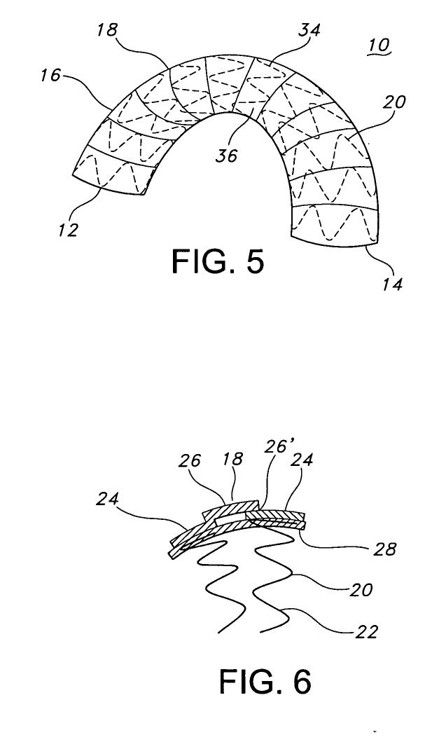

As depicted in FIG. 5, which is a side elevational view of the stent-graft 10 of the present invention, the stent-graft 10 is capable of being highly bent or contoured without kinking of the device and without undesirable deformation of the tubular wall 16. Because of the slidingly juxtaposition of the graft segments 18, these segments move to conform to the curvature of the stent-graft 10. For example, a greater portion 34 of a graft segment 18 may be exposed as compared to a lesser portion 36 to accommodate different curvatures that the stent-graft 10 may experience. Such sliding rearrangement is depicted in FIG. 6. As depicted in FIG. 6, adjacent graft segments 18 may moved away from one and the other to accommodate a bending or otherwise change in shape of the underlying stent 20.

While the stent-graft 10 is depicted in FIG. 5 as having a substantially equal diameter, the present invention is not so limited. For example, the stent-graft 10 may have a varying diameter, for example, having an outwardly or inwardly flared end at either or both of its ends 12, 14. Further, while the stent-graft 10 is depicted as a single lumen device, the present invention is not so limited. For example, the stent-graft 10 may include a tubular branch or branches to define a bifurcated or multi-lumen stent-graft.

The non-textile, polymeric graft segments 18 and/or the second tubular graft structure 28 may suitably be made from extruded, molded or cast polymeric materials. As used herein, the term “textile” refers to a material, such as a yarn, that has been knitted, woven, braided and the like into a structure, including a hollow, tubular structure. As used herein, the term “non-textile” and its variants refer to a material formed by casting, molding, spinning or extruding techniques to the exclusion of typical textile forming techniques, such as braiding, weaving, knitting and the like. Nonlimiting examples of useful polymeric materials for the non-textile polymeric graft portions include polyesters, polypropylenes, polyethylenes, polyurethanes, polynaphthalenes, polytetrafluoroethylenes, expanded polytetrafluoroethylene, silicone, and combinations and copolymers thereof. Desirably, the polymeric material polytetrafluoroethylene (PTFE), including expanded polytetrafluoroethylene (ePTFE).

PTFE exhibits superior biocompatibility and low thrombogenicity, which makes it particularly useful as vascular graft material in the repair or replacement of blood vessels or other bodily lumens. Desirably the non-textile layer is a tubular structure manufactured from ePTFE. The ePTFE material has a fibrous state which is defined by interspaced nodes interconnected by elongated fibrils. The space between the node surfaces that is spanned by the fibrils is defined as the internodal distance. When the term expanded is used to describe PTFE, it is intended to describe PTFE which has been stretched, in accordance with techniques which increase the internodal distance and concomitantly porosity. The stretching may be in uni-axially, bi-axially, or multi-axially. The nodes are spaced apart by the stretched fibrils in the direction of the expansion.

Desirably, the ePTFE material is a physically modified ePTFE tubular structure having enhanced axial elongation and radial expansion properties of up to about 2,000 percent by linear dimension, for example, from about 100 percent by linear dimension to about 2,000 percent by linear dimension, from about 100 percent by linear dimension to about 600 percent by linear dimension, from about 600 percent by linear dimension to about 2,000 percent by linear dimension, and the like. Such expansion properties are not limiting. Such physically modified ePTFE material may be made by reorienting the node and fibril structure through application a radially expansive and longitudinally foreshortening force. The physically modified ePTFE tubular structure is able to be elongated or expanded and then returned to its original state without an elastic force existing therewithin. Additional details of the physically modified ePTFE and methods for making the same can be found in U.S. Pat. No. 6,716,239, the contents of which are incorporated by reference herein.

FIGS. 7-10 depict further details of the undulating stent portion 22 useful with the present invention. As depicted in FIG. 7, peaks 38 of the undulating stent portions 22 may be substantially longitudinally aligned. Further, the undulating stent portions 22 may be longitudinally offset from one and the other by a length, O₁. For example, the peaks 38 of one undulating stent portion 22 may be longitudinally offset from the valleys 40 of an adjacent undulating stent portion 22 by a distance O₁. Any suitable offset length, O₁, may be used with the present invention. Desirably, the offset, O₁, is less than the longitudinal length defined by the longitudinal distance from the peak 38 and the valley 40 of the undulating stent portion 22.

In another aspect, nested undulating stent portions 22 may be useful as the stent 20 of the present invention. As depicted in FIG. 8, the valleys 40 of one undulating stent portion 22 may be longitudinally disposed within a circumferential plane defined by the peaks 38 of an adjacent undulating stent portion 22, which is depicted as offset O₂ in FIG. 8. Desirably, the offset, O₂, is less than the longitudinal length defined by the longitudinal distance from a peak 38 and a valley 40 of the undulating stent portion.

The present invention, however, is not limited to undulating stent portions 22 having longitudinally aligned peaks 38 and valleys 40 as depicted in FIGS. 7-8. For example, as depict in FIG. 9, certain peaks 38 of adjacent undulating stent portions 22 may be proximally disposed relative to one and the other in the longitudinal direction while other peaks 38 are distally disposed to one and the other. As compared to FIG. 7, the peaks 38 and valleys 40 depicted in FIG. 9 are not in substantial longitudinal phase with one and the other. Moreover, as depicted in FIG. 10, the peaks 38 of adjacent undulating stent portions 22 may be offset from the longitudinal axis by an angle, for example by a angle of β₁. The degree of angular offsetting, i.e., β₁ may be suitably varied, for example from about 1° to about 45°, and need not be constant along the longitudinal length of the stent-graft 10 of the present invention. The degree of angular offsetting, i.e., β₁ may be from about 1° to about 20°, more desirably from about 5° to about 20°, for example from about 5° to about 15°.

The undulating stent portions 22 may comprise a single wire 23 or wires 23 that have been helically wound to form the stent 22. The wire 23 or wires 23 at the stent ends 12′, 14′ may be joined by welding, clamping and the like to form an atraumatic end or ends. In other word, the stent 20 of the present invention is desirably free or substantially free of loose wire ends at either or both of the open ends 12, 14. The undulating stent portions 22 of the present invention are not limited to helically wound wire 23 or wires 23, and such undulating stent portions 22 may be formed by other suitable methods. For example, the stent 20 with the undulating stent portions 20 may be machined from a stock of material, including a tubular stock. Such machining may include, without limitation, laser cutting, chemical etching, electrochemical etching, molding, and the like.

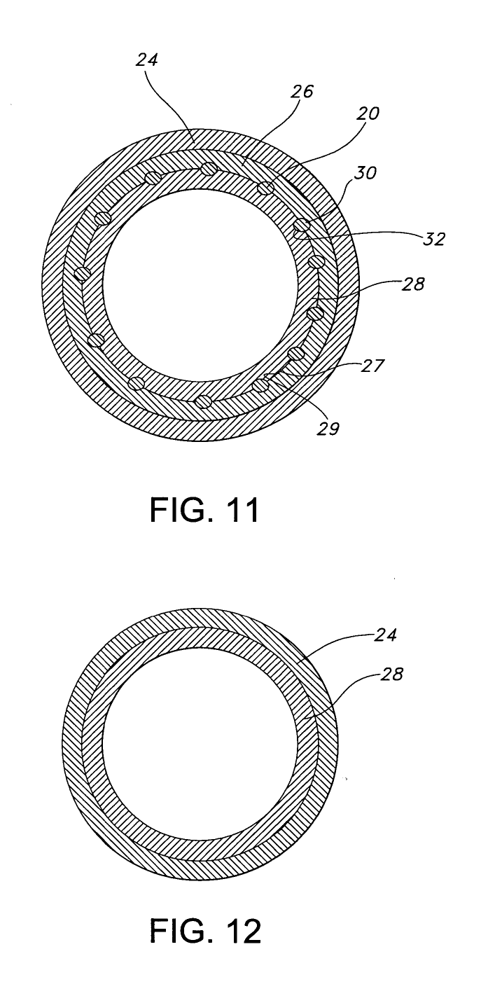

FIG. 11 is a cross-sectional view of the stent-graft of FIG. 2 taken along the 11-11 axis. As depicted in FIG. 11, first portions 24 of the graft segments 18 are secured to exterior surfaces 30 of the stent 20. The second portion 26 of the adjacent graft segment 18 is sliding disposed over the first portion 24. The second polymeric graft structure 28 may be disposed over the luminal surfaces 32 of the stent 22. Additionally, portions 29 of graft structure 28 may be securably disposed to portions 27 of the first portions 24 of the graft segments 18 through the stent interstices.

As depicted in FIG. 12, which is a cross-sectional view of the stent-graft 10 of FIG. 2 taken along the 12-12 axis, not all portions of the second graft layer 28 need be securably attached to the luminal stent surfaces 32. This is especially true where the peaks 38 adjacent undulating stent portion 22 are longitudinally offset in a non-nested fashion, for example, being offset by a length O₁.

The non-textile, polymeric graft portions 24, 28 of the present invention may be secured to one and the other and/or secured to the stent 20 through any suitable means, including, without limitation, lamination, such as heat and/or pressure lamination, and/or adhesive bonding. The bonding agent may include various biocompatible, elastomeric bonding agents such as urethanes, styrene/isobutylene/styrene block copolymers (SIBS), silicones, and combinations thereof. Other similar materials are contemplated. Desirably, the bonding agent may include polycarbonate urethanes sold under the trade name CORETHANE®. This urethane is provided as an adhesive solution with preferably 7.5% Corethane, 2.5 W30, in dimethylacetamide (DMAc) solvent. Details of suitable bonding agents and methods for bonding are further described in U.S. Patent Application Publication Nos. 2003/0017775 A1 and 2004/0182511 A1, the contents of which are incorporated herein by reference.

A method of making the stent-graft 10 of the present invention includes the steps of providing a radially distensible, tubular stent 20 having opposed open ends 12′, 14′ comprising an undulating wire 23 helically wound into a plurality of circumferential windings 22 to define stent wall structure 16′ having opposed exterior and luminal surfaces 30, 32; providing a first non-textile, polymeric graft strip 18; helically winding the first strip 18 over at least one of the circumferential windings 22 of the undulating wire 23 of the exterior stent surface 30 to define a first juxtaposed strip-stent region 25; providing a second non-textile, polymeric graft strip 18; helically winding the second strip 18 over at least one of the circumferential windings 22 of the undulating wire 23 of the exterior stent surface 30 to define a second juxtaposed strip-stent region 25′ and over a portion 24 of the first strip 18 to define an overlap 26′ of the first and the second strips 18; securing portions 24 of the first and second strips 18 at the first and the second juxtaposed regions 25, 25′ to the stent 20; and not securing the graft strips 18 to one and the other at the overlap 26′. The step of securing portions 24 of the first and second strips 18 at the first and the second juxtaposed regions 25, 25′ to the stent 20 may further comprise laminating the strips 18 to the stent regions. The step of not securing the graft strips 18 to one and the other at the overlap 26′ may further comprise masking the strips 18 at the overlap 26′ so that the strips 18 at the overlap 26′ are not laminated to one and the other.

The method for forming the stent-graft 10 may further comprise the steps of providing a non-textile, polymeric tubular graft 28 disposed about the luminal surface 32 of the stent; and securing the graft 28 to the luminal surfaces 32 of the stent 20. This aspect of the method may further comprise the step of securing the graft 28 to the first and the second strips 18.

In one aspect of the present invention, a stent-graft 10 is provided. The stent-graft 10 comprises a radially distensible, tubular stent 20 comprising opposed open ends 12′, 14′ and a stent wall structure 16′ having opposed exterior and luminal surfaces 30, 32; and a segmented, non-textile polymeric tubular structure 16 comprising a plurality of graft segments 18 circumferentially disposed about one of the stent surfaces 30, 32, the graft segments 18 comprising first portions 24 securably disposed to the one the stent surface 30, 32 and second portions 26 not securably disposed to the one the stent surface 30, 32; wherein adjacent graft segments 18 are disposed over one and the other to define overlaps 26′, the overlaps 26′ forming a fluid tight seal when implanted in a body lumen. The second portion 26 of one of the graft segments 18 may be disposed over the first portion 24 of the adjacent graft segment 18. Desirably, the second portion 26 of the graft segment 18 slidingly abuts the first portion 24 of the adjacent graft segment 18. Desirably, the graft segments 18 are disposed over the exterior surfaces 30 of the stent 20.

In this aspect of the present invention, the stent-graft 10 may further comprise a second non-textile polymeric tubular graft structure 28 securably disposed about the luminal surfaces 32 of the stent 20. The first portions 24 of the graft segments 18 may be securably attached to portions of the second graft structure 28. Further, the second portions 26 of the graft segments may not be securably attached to portions of the second graft structure 28.

The stent 20 may further comprise a longitudinal length, wherein the longitudinal length remains substantially constant upon radial expansion or radial contraction of the stent 20. The stent 20 may be a self-expanding stent, a balloon-expandable stent or combinations thereof. Desirably, the stent 20 comprises a plurality of undulating stent segments 22. The stent 20 may comprise an undulating wire 23 helically wound into a plurality of circumferential windings 22 to define the stent wall structure 16′. Desirably, the first portion 24 of the graft segment 18 is securably attached to at least one of the circumferential windings 22 of the undulating wire 23, for example, securably attached to at least two of the circumferential windings 22 of the undulating wire 23. The overlap 26′ of the adjacent graft segments 18 may longitudinally extend over at least one of the circumferential windings 22 of the undulating wire 23. Desirably, the graft segments 18 are substantially unfolded segments.

In another aspect of the present invention, a stent-graft 10 is provided. The stent-graft 10 of this aspect of the present invention comprises a radially distensible, tubular stent 20 having opposed open ends 12, 14 comprising an undulating wire 23 helically wound into a plurality of circumferential windings 22 to define stent wall structure 16′ having opposed exterior and luminal surfaces 30, 32; a non-textile, polymeric tubular graft structure 28 securably disposed about the luminal surface 32 of the stent 20; and a segmented, non-textile polymeric tubular structure 16 comprising a plurality of graft segments 18 circumferentially disposed about the exterior surface 30 of the stent 20, the graft segments 18 comprising first portions 24 securably disposed to the exterior surface 30 of the stent 20 and second portions 26 not securably disposed to the exterior surface 30 of the stent 20; wherein adjacent graft segments 18 are disposed over one and the other to define overlaps 26′, the overlaps 26′ forming a fluid tight seal when implanted in a body lumen.

In another aspect of the present invention, a radially distensible, tubular stent 20 having opposed open ends to define a length therebetween is provided. The stent includes an undulating wire helically wound into a plurality of circumferential windings to define a stent wall structure having opposed exterior and luminal surfaces. Desirably, the undulating wire includes a series of peaks 38 and valleys 40 along its length. Desirably, the peaks may be angularly offset from the longitudinal length of the stent. The peaks may be angularly offset from about 1° to about 45°, desirably from 5° to about 20° from the longitudinal length of the stent.

With any embodiment, the stent-graft 10 may be used for a number of purposes including to maintain patency of a body lumen, vessel or conduit, such as in the coronary or peripheral vasculature, esophagus, trachea, bronchi colon, biliary tract, urinary tract, prostate, brain, and the like. The devices of the present invention may also be used to support a weakened body lumen, or to provide a fluid-tight conduit for a body lumen, or support a weakened or kinked device in a lumen, for example adjunctive use. Adjunctive use involved the deployment of a second device, for example stent-graft 10, to a target site having a device, such as a stent, a graft or stent-graft previously positioned thereat. The stent-graft 10 of the present invention may be used to completely or partially overlap the previous device to alleviate a weakening or a kinking of the previous device, i.e., adjunctive deployment or adjunctive use.

Also, the stent-graft 10 may be treated with any known or useful bioactive agent or drug including without limitation the following: anti-thrombogenic agents (such as heparin, heparin derivatives, urokinase, and PPack (dextrophenylalanine proline arginine chloromethylketone); anti-proliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-miotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promotors (such as growth factor inhibitors, growth factor receptor antagonists, transcriptional activators, and translational promotors); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

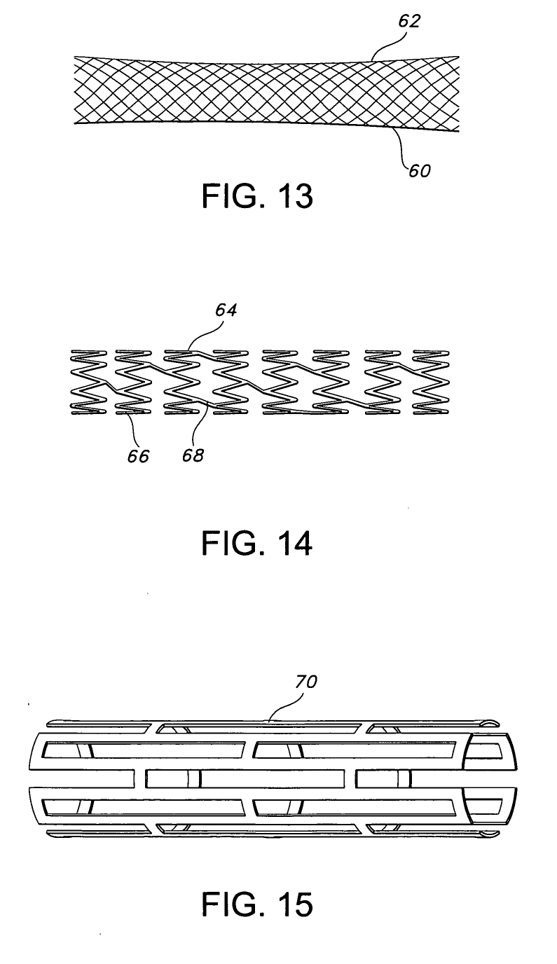

As described above, various stent types and stent constructions may be employed in the invention as the stent 20 in the stent-graft 10. Non-limiting examples of suitable stent geometries for stent 20 are illustrated in FIGS. 13-17. As shown in FIG. 13, wire stent 60 is a hollow tubular structure formed from wire strand 62 or multiple wire strands. Wire stent 60 may be formed by, for example, braiding or spinning wire strand(s) 62 over a mandrel (not shown). Wire stent 60 is capable of being radially compressed and longitudinally extended for implantation into a bodily lumen. The degree of elongation depends upon the structure and materials of the wire stent 60 and can be quite varied, for example, about 5% to about 200% of the length of wire stent 60. The diameter of wire stent 60 may also become several times smaller as it elongates. Unitary stent structures may be obtained by braiding and/or filament winding stent wires to obtain complex stent geometries, including complex stent geometries, including complex bifurcated stents. Alternatively, stent components of different sizes and/or geometries may be mechanically secured by welding or suturing. Additional details of wire stents of complex geometry are described in U.S. Pat. Nos. 6,325,822 and 6,585,758, the contents of which are incorporated herein by reference.

A zig-zag wire stent 64 is also useful as stent 20. Wire strand 66 is being arranged in what can be described as a multiple of “Z” or “zig-zag” patterns to form a hollow tubular stent. The different zig-zag patterns may optionally be connected by connecting member 68. Further, zig-zag wire stent 64 is not limited to a series of concentric loops as depicted in FIG. 14, but may be suitably formed by helically winding of the “zig-zag” pattern over a mandrel (not shown).

A slotted stent 70 is also useful as stent 20. As depicted in FIG. 15, slotted stent 70 is suitably configured for implantation into a bodily lumen (not shown). Upon locating the slotted stent 70 at the desired bodily site, slotted stent 70 is radially expanded and longitudinally contracted for securement at the desired site.

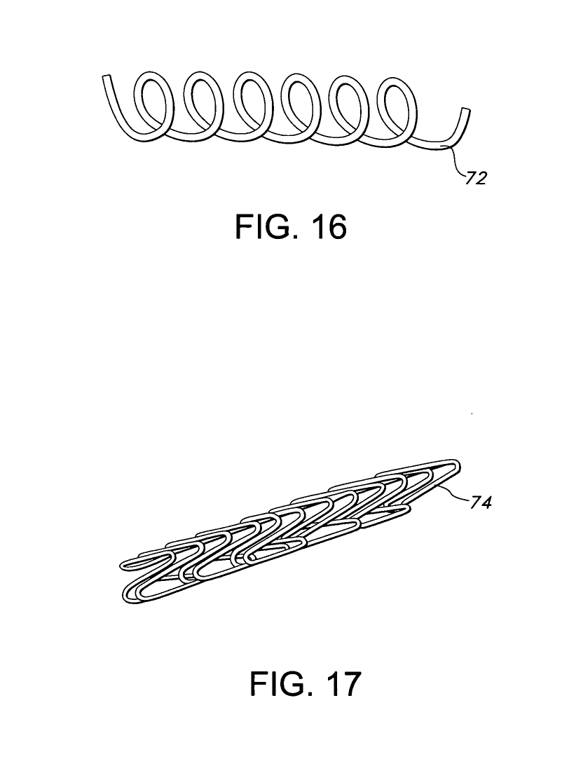

Other useful stents capable of radial expansion are depicted in FIGS. 16 and 17. As depicted in FIG. 16, stent 72 is a helical coil which is capable of achieving a radially expanded state (not shown). Stent 74, as depicted in FIG. 17, has an elongate pre-helically coiled configuration as shown by the waves of non-overlapping undulating windings. These helically coiled or pre-helically stents, commonly referred to as nested stents, are also useful with the practice of the present invention.

The invention being thus described, it will now be evident to those skilled in the art that the same may be varied in many ways. Such variations are not to be regarded as a departure from the spirit and scope of the invention and all such modifications are intended to be included within the scope of the following claims.

What is claimed is:

1. A stent-graft comprising:
a radially distensible, tubular stent comprising opposed open ends to define a length therebetween and a stent wall structure having opposed exterior and luminal surfaces; and
a segmented, non-textile polymeric tubular structure comprising a plurality of graft segments circumferentially disposed about one of said stent surfaces, said graft segments comprising first portions securably disposed to the one said stent surface and second portions not securably disposed to the one said stent surface;
wherein adjacent graft segments are disposed over one and the other to define overlaps, said overlaps forming a fluid tight seal when implanted in a body lumen.

2. The stent-graft of claim 1, wherein said second portion of one of said graft segments is disposed over said first portion of said adjacent graft segment.

3. The stent-graft of claim 2, wherein said second portion of said graft segment slidingly abuts said first portion of said adjacent graft segment.

4. The stent-graft of claim 1, wherein said graft segments are disposed over said exterior surfaces of said stent.

5. The stent-graft of claim 4, further comprising a second non-textile polymeric tubular graft structure securably disposed about said luminal surfaces of said stent.

6. The stent-graft of claim 5, wherein said first portions of said graft segments are securably attached to portions of said second graft structure.

7. The stent-graft of claim 5, wherein said second portions of said graft segments are not securably attached to portions of said second graft structure.

8. The stent-graft of claim 1, wherein said stent further comprises a longitudinal length, wherein said longitudinal length remains substantially constant upon radial expansion or radial contraction of said stent.

9. The stent-graft of claim 1, wherein said stent is a self-expanding stent, a balloon-expandable stent or combinations thereof.

10. The stent-graft of claim 1, wherein said stent comprises a plurality of undulating stent segments.

11. The stent-graft of claim 1, wherein said stent comprises an undulating wire helically wound into a plurality of circumferential windings to define said stent wall structure.

12. The stent-graft of claim 11, wherein said first portion of said graft segment is securably attached to at least one of said circumferential windings of said undulating wire.

13. The stent-graft of claim 11, wherein said first portion of said graft segment is securably attached to at least two of said circumferential windings of said undulating wire.

14. The stent-graft of claim 11, wherein said overlap of said adjacent graft segments longitudinally extends over at least one of said circumferential windings of said undulating wire.

15. The stent-graft of claim 11, wherein said undulating wire comprises a series of peaks and valleys along its length; and further wherein said peaks are substantially longitudinally aligned along the length of said stent.

16. The stent-graft of claim 11, wherein said undulating wire comprises a series of peaks and valleys along its length; and further wherein said peaks are angularly offset from the longitudinal length of said stent.

17. The stent-graft of claim 16, wherein said peaks are angularly offset from about 1° to about 45° from the longitudinal length of said stent.

18. The stent-graft of claim 1, wherein said graft segments are substantially unfolded segments.

19. The stent-graft of claim 1, wherein said graft segments comprise polymeric graft material selected from the group consisting of polyesters, polypropylenes, polyethylenes, polyurethanes, polynaphthalenes, polytetrafluoroethylenes, expanded polytetrafluoroethylenes, silicones, and combinations and copolymers thereof.

20. The stent-graft of claim 1, wherein said graft segments comprise expanded polytetrafluoroethylene.

21. The stent-graft of claim 5, wherein said second tubular graft comprises polymeric graft material selected from the group consisting of polyesters, polypropylenes, polyethylenes, polyurethanes, polynaphthalenes, polytetrafluoroethylenes, expanded polytetrafluoroethylenes, silicones, and combinations and copolymers thereof.

22. The stent-graft of claim 5, wherein said second tubular graft comprises expanded polytetrafluoroethylene.

23. The stent-graft of claim 1, wherein said graft segments are extruded, cast, spun or molded polymeric segments.

24. The stent-graft of claim 1, wherein said stent comprises a biocompatible material selected from the group consisting of metallic materials, polymeric materials, bioabsorbable materials, biodegradable materials, and combinations thereof.

25. A stent-graft comprising:
a radially distensible, tubular stent having opposed open ends to define a length therebetween comprising an undulating wire helically wound into a plurality of circumferential windings to define stent wall structure having opposed exterior and luminal surfaces;
a non-textile, polymeric tubular graft structure securably disposed about said luminal surface of said stent; and
a segmented, non-textile polymeric tubular structure comprising a plurality of graft segments circumferentially disposed about said exterior surface of said stent, said graft segments comprising first portions securably disposed to said exterior surface of said stent and second portions not securably disposed said exterior surface of said stent;
wherein adjacent graft segments are disposed over one and the other to define overlaps, said overlaps forming a fluid tight seal when implanted in a body lumen.

26. The stent-graft of claim 25, wherein said undulating wire comprises a series of peaks and valleys along its length; and further wherein said peaks are substantially longitudinally aligned along the length of said stent.

27. The stent-graft of claim 25, wherein said undulating wire comprises a series of peaks and valleys along its length; and further wherein said peaks are angularly offset from the longitudinal length of said stent.

28. The stent-graft of claim 27, wherein said peaks are angularly offset from about 1° to about 45° from the longitudinal length of said stent.

29. A method of making a stent-graft comprising:
providing a radially distensible, tubular stent having opposed open ends comprising an undulating wire helically wound into a plurality of circumferential windings to define stent wall structure having opposed exterior and luminal surfaces;
providing a first non-textile, polymeric graft strip;
helically winding said first strip over at least one of said circumferential windings of said undulating wire of said exterior stent surface to define a first juxtaposed strip-stent region;
providing a second non-textile, polymeric graft strip;
helically winding said second strip over at least one of said circumferential windings of said undulating wire of said exterior stent surface to define a second juxtaposed strip-stent region and over a portion of said first strip to define an overlap of said first and said second strips;
securing portions of said first and second strips at said first and said second juxtaposed regions to said stent; and
not securing said graft strips to one and the other at said overlap.

30. The method of claim 29, wherein the step of securing portions of said first and second strips at said first and said second juxtaposed regions to said stent further comprises laminating said strips to said stent regions.

31. The method of claim 30, wherein the step of not securing said graft strips to one and the other at said overlap comprises masking said strips at said overlap so that said strips at said overlap are not laminated to one and the other.

32. The method of claim 29, further comprising:
providing a non-textile, polymeric tubular graft disposed about said luminal surface of said stent; and
securing said graft to said luminal surfaces of said stent.

33. The method of claim 32, further comprising:
securing said graft to said first and said second strips.

34. A radially distensible, tubular stent having opposed open ends to define a length therebetween comprising an undulating wire helically wound into a plurality of circumferential windings to define stent wall structure having opposed exterior and luminal surfaces; wherein said undulating wire comprises a series of peaks and valleys along its length; and further wherein said peaks are angularly offset from the longitudinal length of said stent.

35. The stent-graft of claim 34, wherein said peaks are angularly offset from about 1° to about 45° from the longitudinal length of said stent.

36. A method of supporting a first stent-graft deployed within a bodily lumen, comprising:
providing a second stent-graft comprising a radially distensible, tubular stent comprising opposed open ends to define a length therebetween and a stent wall structure having opposed exterior and luminal surfaces; and a segmented, non-textile polymeric tubular structure comprising a plurality of graft segments circumferentially disposed about one of said stent surfaces, said graft segments comprising first portions securably disposed to the one said stent surface and second portions not securably disposed to the one said stent surface; wherein adjacent graft segments are disposed over one and the other to define overlaps, said overlaps forming a fluid tight seal when implanted in a body lumen;
deploying the second stent-graft within the first stent-graft; and
expanding the second stent-graft to support the first stent-graft.
